(19) United States
(12) Patent Application Publication
(10) Pub. No.: US 2011/0262552 A1
(43) Pub. Date: Oct. 27, 2011

(54) Compositions Comprising Plant Extracts and Use Thereof for Treating Inflammation

(76) Inventors: Guy Chamberland, Boucherville (CA); Celine Devaux, Boucherville (CA); Peter Bollen, Drongen (BE)

(21) Appl. No.: 13/120,739

(22) PCT Filed: Sep. 25, 2009
(86) PCT No.: PCT/CA2009/001360

§ 371 Date: Jul. 11, 2011

Related U.S. Application Data
(60) Provisional application No. 61/102,106, filed on Oct. 2, 2008.

(51) Int. Cl. A61K 35/56; A61K 33/34; A61P 21/02; A61P 29/00; A61P 19/02; A61K 36/45; A61K 33/42

(52) U.S. Cl. 424/547; 424/732; 424/637; 424/602

(57) ABSTRACT

Provided are compositions comprising Harpagophytum procumens (Devil's claw) root extract and Ribes nigrum (Black Currant) leaf or seed extract. Other components may also be included. Also provided are methods of preventing or treating inflammation or a condition that results from inflammation.

### FIELD OF INVENTION

The present invention relates to anti-inflammatory compositions. More specifically, the present invention relates to anti-inflammatory plant extract compositions.

### BACKGROUND OF THE INVENTION

Inflammation is a self-defensive reaction designed to eliminate or neutralize potentially damaging stimuli and restore tissue integrity following insult (1). Inflammation can also be a two-edged sword. Pro-inflammatory mediators usually play a role in controlling important physiological functions, such as the regulation of blood pressure, platelet aggregation and body-temperature. In acute conditions, such as infection, inflammation protects tissue against invading pathogens and promotes healing. However, under pathological conditions these normally protective immune responses can be erroneously misdirected to damage the body's own tissues. This activity can lead to a plethora of adverse outcomes, ranging from localized swelling and discomfort to organ failure (2, 3).

Pathological immune responses are especially evident in conditions with prominent autoimmune etiologies, such as arthritis. In fact, the term arthritis literally means inflammation of the joint. While often referred to as a single disease, arthritis is a general term that describes more than a hundred medical conditions that affect 4.6 million adults and 30,000 children in Canada alone (4). While the most common form of arthritis, osteoarthritis, is prevalent in people over 60, arthritis in its various forms can start as early as infancy.

Rheumatoid arthritis is an inflammatory joint disease that involves destruction of the extracellular matrices of articular cartilage and bone (5). The underlying disturbance in immune regulation that is responsible for the localized joint pathology in rheumatoid arthritis results in the release of inflammatory mediators in the synovial fluid and synovium that directly and indirectly influence cartilage homeostasis (6). Both rheumatoid arthritis and osteoarthritis are characterized by inflammation of the musculoskeletal system and specifically the joints which can lead to pain, stiffness, and damage to joint cartilage and surrounding structures. Such damage can lead to joint weakness, instability and visible deformities that, depending on the location of joint involvement, can interfere with the most basic motor skills.

Arthritis is by no means a condition restricted to humans. The selective breeding of companion and domestic animals, such as dogs and cats, has inadvertently led to the propagation of many autoimmune and inflammatory diseases, including arthritis. For example, twenty-percent of the canine and feline population greater than one year old is reported to have some degree of osteoarthritis (7). Multiple etiologies have been suspected of contributing to the formation of the disease, including defective articular cartilage structure and biosynthesis, joint trauma, joint instability, congenital and developmental abnormalities, and inflammatory conditions.

Management of arthritis in subjects typically involves a multimodal approach that includes one or more of the following: activity control; weight management; nutritional support; physical therapy; and administration of nonsteroidal anti-inflammatory drugs (NSAIDs), corticosteroids, analgesic medications, and nutraceuticals (7-9).

During the past decade, numerous therapeutic agents have been introduced and used for the treatment of arthritis in subjects with various levels of effectiveness. The application of these therapies is further complicated by wide variations in their documented efficacy and safety. NSAIDs, such as aspirin and phenylbutazone, have historically been the most commonly used agents as a means of reducing prostaglandin synthesis (primarily PGE2) through inhibition of cyclooxygenase (COX) (8, 9). However, complications such as gastric ulceration have seen the use of these agents decline in favour of safer alternatives. The use of corticosteroids in the treatment of OA is controversial because, although they reduce synovitis and inflammatory changes in the cartilage, they are detrimental to cartilage health by decreasing proteoglycan and collagen production. Intra-joint injections of corticosteroids may lessen systemic side effects, but is associated with steroid arthropathy. Presently, it is difficult to make recommendations as to which treatment is best for subjects because few head-to-head comparisons of these products have been made, emphasizing the need for continued well-designed experimental and clinical research to evaluate their efficacy and safety.

There is a need in the art to identify novel anti-inflammatory compositions. Further, there is a need in the art to identify anti-inflammatory, plant extract compositions that can be used separately or in combination with existing therapies to prevent or treat inflammation.

### SUMMARY OF THE INVENTION

The present invention relates to anti-inflammatory compositions. More specifically, the present invention relates to anti-inflammatory plant extract compositions.

According to the present invention there is provided a composition comprising Harpagophytum procumens (Devil's claw) root extract and Ribes nigrum (Black Currant) leaf and/or seed extract.

The present invention also provides a composition as described above wherein the Harpagophytum procumens (Devil's claw) root extract comprises about 5% to 20% harpagosides (as measured by UV-VIS spectrometry analysis), and the Ribes nigrum leaf and/or seed extract comprises between about 0.1% to 2% rutines (as measured by HPLC Diode Array).

The present invention also provides a composition as described above, wherein the composition further comprises one or more components or extracts of Perna canaliculus (Green Shell Mussel), Salix Alba (White Willow) plant and/or bark, Tanacetum Parthenium (Feverfew) herb and/or flower, Equisetum arvense (Horsetail), Spireae ulmaria (propwort), Betula alba (Birch), Urtica dioica (Stinging Nettle), Solidago virgaurea (Goldenrod), Bosellia seratta (Boswellia), Curcumin longa (Tumeric), Bromelain (Ananas comosus), Griffonia simplicifolia, Petasites hybridus (Butterbur), marine algae, or a combination thereof.

The present invention also provides a composition as described above, wherein the composition further comprises fish meal, type II collagen, glucosamine, DHA, EPA, hyaluronic acid, L-glutamine, chondroitin, methylsulfonylmethane, dextrose, whey protein, minerals including, without limitation calcium, phosphate, manganese, magnesium, zinc, copper (free, chelated or both), vitamin B12, vitamin E, Vitamin C, beta carotene, microcrystalline cellulose, polyethylene glycol, starch, stearin, hydrogenated oils, talc, stearate or a combination thereof.

The present invention also provides a composition as described above comprising Harpagophytum procumbens (Devil's claw) root extract, Ribes nigrum (Black currant) leaf and/or seed extract, Perna canaliculus (Green Shell Mussel), Salix Alba (White Willow) plant and/or bark extract, and Tanacetum Parthenium (Feverfew) herb and/or flower extract.

The present invention also provides a composition as described above comprising about 250 mg Harpagophytum procumens (Devil's claw) root extract standardized to about 10% harpagosides (as measured by UV-VIS spectrophotometry), about 100 mg Ribes nigrum (Black currant) leaf or seed extract standardized to about 1% rutines, about 250 mg Perna canaliculus (Green-Shell mussel) standardized to about 20% omega-3 polyunsaturated fatty acids, about 100 mg Salix alba standardized to about 10% salicin and Tanacetum parthenium (Feverfew) herb and/or flower extract standardized to about 0.2% parthenolide.

The present invention also provides a composition as described above and further comprising Salix alba (White Willow) plant and/or bark extract, Tanacetum Parthenium (Feverfew) herb and/or flower extract, Boswellia seratta (Boswellia) and optionally DHA, EPA or both.

The present invention also provides a composition as described above further comprising Boswellia seratta (Boswellia), DHA-EPA, Cucurma longa (Tumeric), Bromelain (Ananas comosus) and optionally one or more of hyaluronic acid, L-glutamine, chondroitin, methylsulfonylmethane and glucosamine.

The present invention also provides a composition as described above further comprising fish meal/collagen type II, Glucosamine sulphate, marine algae, methylsulfonylmethane, chondroitine sulphate, Bromelia (pineapple extract), curcumin, L-glutamine, hyaluronic acid, dextrose, DHA complex, whey protein, manganese chelate, manganese sulphate, zinc chelate, Zinc oxide, Vitamin B12, Vitamin B6, Vitamin B9, Copper chelate, Cupric sulphate, Vitamin E, Vitamin C, Beta carotene, Dextrose, Equisetum arvensis, Boswellia serata, Harpagophytum procumbens, and Ribes nigrum.

The present invention also provides a composition as described above comprising Devil's claw, Green shell mussel powder, Ribes nigrum, Viola tricolour, Zea maize (corn silk), Origanum vulgare, Dicalcium phosphate, Microcrystalline cellulose E460, Polyethylene glycol, Starch, Stearin, Glyceryl dibehenate, Hydrogenated cottonseed oil, Talc, and Magnesium stearate.

The present invention also provides a method of preventing, treating or both preventing or treating inflammation or a condition that results from inflammation in a subject comprising the step of administering an anti-inflammatory composition as described herein to the subject in need thereof.

The present invention also provides a method as described above, wherein the inflammation or condition that results from inflammation is arthritis.

The present invention also provides a method as described above, wherein the arthritis is rheumatoid arthritis or osteoarthritis.

The present invention also provides a method as described above, wherein the subject is concurrently treated with a steroid, non steroidal anti-inflammatory drug, narcotic, muscle relaxant or any combination thereof. Alternatively, a single product that contains both the steroid, NSAID, other anti-inflammatory agent, etc, may be formulated in the plant extract formulation as described herein.

The present invention also provides a method as described above, wherein the condition that results from inflammation is pain.

The present invention also provides a method as described above wherein the subject is a mammalian subject. In a further embodiment the subject may be a human subject.

This summary of the invention does not necessarily describe all features of the invention.

### DETAILED DESCRIPTION

The following description is of a preferred embodiment.

According to the present invention, there is provided a composition comprising Harpagophytum procumens (Devil's claw) root extract and Ribes nigrum (Black Currant) leaf or seed extract. Other components may also be included as described herein and throughout.

In a preferred embodiment, the Harpagophytum procumens root extract comprises between about 5% to 30% harpagosides, more preferably about 10% to 20% harpagosides, for example but not limited to 5%, 7%, 10%, 12%, 15%, 17%, 19%, 21%, 22%, 23%, 25%, 27%, 29%, 30% or any value therein between. The amount of harpagosides may also be defined by a range of any two of the values listed above or any value therein between. Further, in a preferred embodiment, the Ribes nigrum leaf and/or seed extract comprises between about 0.1% to 5% rutines, for example, but not limited to 0.1%, 0.5%, 0.7%, 1%, 1.5%, 2%, 2.5%, 3%, 3.5%, 4%, 4.5%, 5% or any value therein between. More preferably, rutines are present between about 0.5% to 3%, even more preferably about 1-2%. The amount of rutines may also be defined by a range of any two of the values listed above or any value therein between. The present invention also contemplates compositions comprising components outside the ranges provided above.

The plant extract composition may also comprise additional plant components or extracts, for example, but not limited to, one or more components or extracts of Perna canaliculus (Green Shell Mussel), Salix Alba (White Willow) bark and/or plant extract, Tanacetum Parthenium (Feverfew) herb and/or flower, Equisetum arvense (Horsetail), Spireae ulmaria (propwort), Betula alba (Birch), Urtica dioica (Stinging Nettle), Solidago virgaurea (Goldenrod), Bosellia seratta (Boswellia), Curcumin longa (Tumeric), Bromelain (Ananas comosus), Griffonia simplicifolia, Petasites hybridus (Butterbur), marine algae, or a combination thereof.

Further, it is contemplated that the composition comprising plant extracts may comprise one or more additional non-plant components, for example, but not limited to fish meal, type II collagen, glucosamine, docosahexaenoic acid (DHA), eicosapentaenoic acid (EPA), hyaluronic acid, L-glutamine, chondroitin, methylsulfonylmethane, dextrose, whey protein, minerals including, without limitation calcium, phosphate, manganese, magnesium, zinc, copper (free, chelated or both), vitamin B12, vitamin E, Vitamin C, beta carotene, cellulose, microcrystalline cellulose, polyethylene glycol, starch, silicon dioxide, stearin, talc, stearate, maltodextrin, glycerol Bihenate, hydrogenated oil, for example, but not limited to hydrogenated cotton seed oil, one or more flavoring agents, for example, but not limited to vanillin, or any combination thereof.

In an embodiment of the present invention, the composition comprises Harpagophytum procumbens (Devil's claw) root extract, Ribes nigrum (Black Currant) leaf and/or seed extract, Perna canaliculus (Green Shell Mussel), Salix Alba (White Willow) bark and/or plant extract, and Tanacetum Parthenium (Feverfew) herb and/or flower extract.

In the above composition, it is generally preferred that Harpagophytum procumens root extract comprises between about 5% to 20%, more preferably about 10% harpagosides; Ribes nigrum leaf and/or seed extract comprises between about 0.1% to 2% rutines, more preferably about 1% rutines; Perna canaliculus (Green-Shell mussel) comprises between about 10% to 30% omega-3 polyunsaturated fatty acids, preferably about 20% omega-3 polyunsaturated fatty acids; Salix alba comprises about 0.5% to about 20% salicin, preferably about 10% salicin; Tanacetum Parthenium (Feverfew) comprises 0.1% to 0.5% parthenolide, preferably 0.2% parthenolide. The present invention also contemplates compositions comprising components outside the ranges provided above.

Without wishing to be limiting in any manner, the composition comprising plant extracts is formulated into a suitable oral dosage form, for example, but not limited to a powder that may be consumed as such or after mixing with a suitable liquid. More preferably, the composition is formulated into pills, tablets or capsules for oral consumption. For example, but not wishing to be limiting in any manner, capsules comprising 250 mg Harpagophytum procumens (Devil's claw) root extract standardized to comprise about 10% harpagosides, 100 mg Ribes nigrum (Black currant) leaf or seed extract standardized to comprise about 1% rutines, 250 mg Perna canaliculus (Green-Shell mussel) standardized to comprise about 20% omega-3 polyunsaturated fatty acids, 100 mg Salix alba standardized to comprise about 10% salicin and Tanacetum parthenium (Feverfew) extract standardized to comprise about 0.2% parthenolide are prepared using standard procedures known in the art. The composition may also comprise silicon and or other components, for example, but not limited to from Equisetum. In an embodiment, which is not meant to be limiting in any manner, the composition comprises about 50 mg Equisetum that is standardized to comprise about 7-15% silicon.

The present invention also contemplates a composition comprising about 240 mg Harpagophytum procumbens (Devil's claw) root extract standardized to about 10% harpagosides (as measured using UV-VIS spectrophotometry detection), about 60 mg Ribes nigrum (Black currant) leaf or seed extract standardized to about 1% rutines, about 50 mg Salix alba (White Willow) plant and/or bark extract standardized to about 10% salicin, about 50 mg Tanacetum Parthenium (Feverfew) herb or flower extract standardized to about 0.2% parthenolide, about 240 mg Boswellia seratta (Boswellia) extract standardized to a minimum of about 69% boswellic acids and optionally about 40 mg of DHA, EPA or both.

In a further embodiment of the present invention, there is provided a plant extract composition comprising about 60 mg Harpagophytum procumbens (Devil's claw) root extract standardized to about 10% harpagosides (as measured using UV-VIS spectrophotometry detection), about 60 mg Ribes nigrum (Black Currant) leaf or seed extract standardized to about 1% rutines, about 180 mg Boswellia seratta (Boswellia) extract standardized to a minimum of 69% boswellic acids, about 40 mg DHA-EPA, about 35 mg Cucurma longa (Tumeric) extract standardized to a minimum of about 95% curcumin, about 40 mg Bromelain (Ananas comosus) extract standardized to a minimum of about 2000-2500 GDU (Gelatin Dissolving Units) and optionally one or more of hyaluronic acid (minimum of about 15 mg), L-glutamine (about 30 mg), chondroitin (about 60 mg or more), methylsulfonylmethane (about 90 mg or more), glucosamine (about 300 mg or more) or any combination thereof.

In a further embodiment, there is provided a composition comprising Harpagophytum procumbens (Devil's claw) root extract, Ribes nigrum (Black Currant) leaf or seed extract, Fish meal/collage type II, Glucosamine sulphate, marine algae, methylsulfonylmethane, chondroitine sulphate, Bromelia (pineapple extract), Curcumin, L-glutamine, hyaluronic acid, dextrose, DHA, Whey protein, Manganese chelate, Manganese sulphate, Zinc chelate, Zinc oxide, Vitamin B12, Vitamin B6, Vitamin B9, Copper chelate, Cupric sulphate, Vitamin E, Vitamin C, Beta carotene, Dextrose, Equisetum arvensis, and Boswellia serata.

In a further embodiment, there is provided a composition comprising Harpagophytum procumbens (Devil's claw), Green shell mussel powder, Ribes nigrum, Viola tricolour, Zea maize, Origanum vulgare, Dicalcium phosphate, Microcrystalline cellulose E460, Polyethylene glycol, Starch, Stearin, Glyceryl dibehenate, Hydrogenated cottonseed oil, Talc and Magnesium stearate.

The present invention also contemplates a method of preventing and/or treating inflammation or conditions that result from inflammation, for example, but not limited to arthritis and the like by administering an anti-inflammatory plant extract composition as described herein to a subject in need thereof. As would be understood by a person of skill in the art, arthritis is meant to include rheumatoid arthritis and osteoarthritis, but is not limited to only these conditions. In a preferred embodiment, which is not meant to be limiting in any manner, it is generally preferred that an anti-inflammatory plant extract composition, as described herein, be administered daily for at least two weeks, more preferably four weeks to prevent and/or treat inflammation or conditions that result from inflammation.

The present invention also contemplates a method of preventing and/or treating inflammation or conditions that result from inflammation, for example, but not limited to arthritis and the like by combined therapy of:

a) administering an anti-inflammatory plant extract composition as described herein, and

b) administering a further anti-inflammatory therapy to the subject in need thereof,

or;

c) administering a single anti-inflammatory product that comprises an anti-inflammatory plant extract composition as described herein plus a further anti-inflammatory therapy such as an NSAID, steroid, analgesic compound or any combination thereof.

The further anti-inflammatory therapy (as recited in parts b,c above) may comprise any therapy that is indicated or prescribed for the treatment of inflammation or inflammatory conditions, for example administration of one or more analgesics, steroids, or non-steroidal anti-inflammatory drugs (NSAIDS) such as, without limitation, nabumetone, Celecoxib, Rofecoxib, Valdecoxil, Lumiracoxib, Etoricoxib, Naproxen, Indomethacin, Diclofenac, Meloxicam, Nimesulide, ibuprofen, 6-MNA, acetaminophen, aspirin, Deracoxib, Firocoxib, Etodolac, Meloxicam, Carprofen, Tepoxalin, or other drugs, including, without limitation narcotics, synthetic drugs, muscle relaxants or the like. As described, the further anti-inflammatory therapy may be administered concurrently or separately from the step of administering an anti-inflammatory plant extract composition. However, it is generally preferred that the two therapies are administered concurrently (or as a single formulation that contains the two therapies) as the use of an anti-inflammatory plant extract composition may reduce the amount or extent of further anti-inflammatory therapy needed. For example, anti-inflammatory plant extract compositions that reduce the amount of NSAIDS required to alleviate the pain associated with an arthritic condition, may reduce undesirable side effects associated with NSAID therapy, for example, stomach bleeding, ulcers, constipation and the like.

It is also contemplated that the further anti-inflammatory therapy may comprise compression, elevation of an affected area, and/or the application of heat and/or ice to an affected area.

The present invention will be further illustrated in the following examples.

### EXAMPLES

TABLE I
Representative Anti-Inflammatory Compositions 1-4:

| Anti- | | |
|---|---|---|
| inflam- | | |
| matory | | Amount of |
| Compo- | | respective |
| sition # | Components | components |
| 1 | 1 Harpagophytum procumbens:1 Ribes | 125 + 125 mg |
| | nigrum | |
| 2 | 1 Harpagophytum procumbens:1 Ribes | 83.3 + 83.3 + |
| | nigrum:1 Green Shell Mussel | 83.3 mg |
| 3 | 1 Harpagophytum procumbens:1 Ribes | 50 + 50 + 50 + |
| | nigrum:1 Green Shell Mussel:1 Viola | 50 + 50 mg |
| | tricolour:1 Origanum vulgare | |
| 4 | 1 Harpagophytum procumbens:1 Ribes | 62.5 + 62.5 + |
| | nigrum:½ Bromellian:½ Curcumin:1 | 31.25 + 31.25 + |
| | Boswellia serrata | 62.5 mg |

TABLE II
Representative Anti-Inflammatory Composition 5:

| | Amount in |
|---|---|
| Ingredient List | 60 grams (g) |
| Harpagophytum procumbens | 2.25 |
| (Devil's claw) | |
| Ribes nigrum (Black currant) | 2.25 |
| Fish meal/collagen type II | 8.16 |
| Glucosamine sulphate | 7.20 |
| Calcareous marine algae | 2.70 |
| Methylsulfonylmethane | 6.00 |
| Chondroitine sulphate | 2.64 |
| Bromelia (pineapple extract) | 2.40 |
| Curcumin | 1.98 |
| L-glutamine | 1.62 |
| Hyaluronic acid | 0.12 |
| Flavour | 0.18 |
| Dextrose | 3.52 |
| DHA complex | 2.16 |
| Whey protein | 3.4 |
| Manganese chelate | 0.96 |
| Manganese sulphate | 0.73 |
| Zinc chelate | 0.96 |
| Zinc oxide | 0.67 |
| Vitamin B12 | 0.024 |
| Vitamin B6 | 0.024 |
| Vitamin B9 | 0.03 |
| Copper chelate | 0.21 |
| Cupric sulphate | 0.21 |
| Vitamin E | 0.55 |
| Vitamin C | 3.79 |
| Beta carotene | 0.29 |
| Dextrose | 0.47 |
| Equisetum arvensis | 2.25 |
| Boswellia serata | 2.25 |

TABLE III
Anti-inflammatory Composition 6:

| | Amount in |
|---|---|
| Ingredient List | 14 gram (g) |
| Harpagophytum procumbens | 0.75 |
| (Devil's claw) | |
| Ribes nigrum (Black Currant) | 0.75 |
| Green shell mussel powder | 4.50 |
| Viola tricolour | 1.00 |
| Zea maize | 1.50 |
| Origanum vulgare | 1.50 |
| Dicalcium phosphate | 1.50 |
| Microcrystalline cellulose E460 | 0.65 |
| Polyethylene glycol | 0.50 |
| Starch | 0.50 |
| Stearin | 0.50 |
| Glyceryl dibehenate | 0.15 |
| Hydrogenated cottonseed oil | 0.10 |
| Talc | 0.05 |
| Magnesium stearate | 0.05 |

TABLE IV
Representative Anti-inflammatory Composition 7:

| Ingredient | Amount |
|---|---|
| Harpagophytum procumens (Devil's | 250 mg |
| claw) root extract standardized to about | |
| 10% harpagosides | |
| Ribes nigrum (Black currant) leaf/seed | 100 mg |
| extract standardized to about 1% rutines | |
| Perna canaliculus (Green-Shell mussel) | 250 mg |
| standardized to about 20% omega-3 | |
| polyunsaturated fatty acids | |
| Salix alba extract standardized to about | 100 mg |
| 10% salicin | |
| Tanacetum parthenium (Feverfew) bark | 100 mg |
| Equisetum arvense (Horsetail) | 50 mg |

In a preferred embodiment, Harpagophytum procumbens root is extracted with alcohol, preferably ethanol, and the amount of harpagosides is determined by photometric analysis as known in the art. Other methods also may be used to determine the amount of harpagosides in the composition. As will be understood by a person of skill in the art, different measuring techniques may result in different amounts of components being determined in the composition. For example, but not to be considered limiting in any manner, HPLC analysis of an extract of Harpagophytum procumbens root indicated about 2.7% harpagosides while UV-VIS spectrophotometric analysis indicated about 10% harpagosides. In the examples provided herein, the amount of harpagosides recited in the composition has been determined by photometric analysis unless stated otherwise. Representative methods of determining harpagosides are known in the art (Günther M, Schmidt P C.; J Pharm Biomed Anal. 2005 Apr. 1; 37(4):817-21; Comparison between HPLC and HPTLC-densitometry for the determination of harpagoside from Harpagophytum procumbens CO(2)-extracts.)

Ribes nigrum leaf and/or seed is extracted with alcohol, preferably ethanol, and the amount of rutines is preferably determined by HPLC Diode array analysis as known in the art, or any other appropriate method as is known in the art, for example, but not limited to as described by Santagati N A, Salerno L, Attaguile G, Savoca F, Ronsisvalle G.; J Chromatogr Sci. 2008 February; 46(2):150-6.; Simultaneous determination of catechins, rutin, and gallic Acid in cistus species extracts by HPLC with diode array detection.

Green Shell mussel is preferably provided as a freeze dried and/or ground powder.

Salix Alba (bark, plant or combination thereof) is preferably extracted with alcohol, more preferably ethanol. Salicin content may be determined by photometric analysis or another appropriate method known in the art. Extracts may comprise salicin in an amount generally between about 0.001% to 20%, for example, but not limited to 0.001%, 0.01%, 0.1%, 1%, 2%, 3%, 4%, 5%, 7%, 9%, 10%, 12%, 15%, 17%, 20%, or any value or range of values between the values indicated above. Representative methods of for making such determinations are known in the art, for example, but not limited to ChromaDex Analytical Method ‘Determination of Salicin in Capsules Containing Willow Bark Extract by HPLC’ (CD-ATM-024-03-00).

Curcumin longa (tumeric) is preferably extracted with acetone, alcohol or a combination thereof, more preferably acetone, ethanol or a combination thereof. Curcumin content may be determined by photometric analysis or another appropriate method known in the art. Typical extracts comprise greater than about 80% curcumins. Representative methods of for making such determinations are known in the art, for example, but not limited to Pak Y, Patek R, Mayersohn M.; J Chromatogr B Analyt Technol Biomed Life Sci. 2003 Nov. 5; 796(2):339-46. Sensitive and rapid isocratic liquid chromatography method for the quantitation of curcumin in plasma.

Boswellia seratta is preferably extracted with alcohol, for example ethanol or the like and extracts thereof typically comprise greater than about 69% boswellic acids. Representative methods of for making such determinations are known in the art, for example, but not limited to Shah S A, Rathod I S, Suhagia B N, Pandya S S, Parmar V K.; J Chromatogr Sci. 2008 September; 46(8):735-8.; A simple high-performance liquid chromatographic method for the estimation of boswellic acids from the market formulations containing Boswellia serrata extract.

Tanacetum parthenium (herbs and/or flowers) is preferably extracted with alcohol, more preferably ethanol or the like and extracts thereof typically comprise between about 0.2% to 0.5% parthenolide.

Equisetum arvense (aerial parts) is preferably extracted with water and/or alcohol, for example, but not limited to ethanol or methanol. Extracts typically comprise between about 5% to 20% silica.

Spirea ulmaria is preferably extracted with alcohol, water, acetone or a combination thereof. Preferably the extraction solvent is ethanol.

Betula alba leaf is preferably extracted with alcohol, water, acetone or a combination thereof. Preferably the extraction solvent is ethanol.

Urtica dioica (leaf, root or combination thereof) is preferably extracted with a solvent or solvent system comprising for example alcohol, hexane, water, acetone or a combination thereof. Preferably the extraction solvent is ethanol or a combination of ethanol and water.

Solidago virgaurea is preferably extracted with alcohol. Preferably the extraction solvent is ethanol.

Bromelain is preferably extracted from pineapple fruit core by mechanical procedures, for example, but not limited to homogenization or the like under cold temperatures. The mechanical procedures release intracellular enzyme. The mixture is centrifuged and enzyme and other components are precipitated from the resulting solution by ammonium sulfate precipitation, for example but not limited to by addition of 55% ammonium sulfate solution. The precipitated bromelain is dissolved in a buffer, for example, but not limited to 0.02M acetate buffer, pH 4.8 and then lyophilized. Other methods of extracting bromelain as known in the art also may be employed.

Griffonia simplicifolia (seed) are preferably ground into a powder and extracted using a water-methanol mixture and heating the extraction vessel while ultrasonicating. The resultant solution is filtered through a 0.45 microlitre filter and the liquid is lyophized. Alternatively, 5-HTP may be isolated by chromatography. Other methods of extracting Griffonia simplicifolia seed are also contemplated.

Petasites hybridus (rhizome) is preferably extracted using alcohol, for example, but not limited to ethanol, methanol or a combination thereof.

Viola Tricolour is preferably extracted using water, acetone, alcohol or a combination thereof. In an embodiment that is not meant to be limiting in any manner, the plant is extracted with ethanol or a mixture of ethanol and water.

Origanum Vulgare is preferably extracted using acetone, water, alcohol or a combination thereof. In an embodiment that is not meant to be limiting in any manner, the plant is extracted with ethanol or a mixture of ethanol and water.

Zea Maize (corn silk) is preferably extracted using acetone, water or alcohol. In an embodiment that is not meant to be limiting in any manner, the plant is extracted with ethanol or a mixture of ethanol and water.

In general, and without wishing to be limiting in any manner, an extract of a plant may be produced by mechanically processing the plant or plant part into a powder or fine particles for extraction. This step may be performed with or without extraction solvent and/or other solvents. Preferably the extraction process is performed on ice or under cold temperatures unless otherwise stated to preserve the active components of the plants. The extraction solvent may be removed and optionally filtered and the residual solvent may be concentrated as known in the art, for example, but not wishing to be limited to evaporation, spray drying, hydrodistillation, lyophilization, nebulization or other method known in the art. The final extraction products are preferably dry powders that may be used as necessary to produce the compositions as described herein.

### Example 1

### Carrageenin-Induced Acute Inflammation in Mice

Phenylbutazone, Celebrex, Naproxen and individual plant extract compositions as described in Table V were suspended/dissolved in a vehicle of 2% Tween 80.

Male CD-1 (Crl.) derived mice weighing 22±1 g were obtained from BioLasco Taiwan (under Charles River Laboratories Technology Licensee). All animals were housed in a controlled temperature (22° C.-24° C.) and humidity (60%-80%) environment with 12 hours light/dark cycles for at least one week prior to experimental use. The animals had free access to standard lab chow for mice (MF-18 (Oriental Yeast Co., Ltd., Japan) and Reverse Osmosis water was granted. All aspects of the study including housing, experimentation and disposal of animals were performed in general accordance with the Guide for the Care and Use of Laboratory Animals (National Academy Press, Washington, D.C., 1996).

Groups of 5 CD-1 male mice were fasted overnight prior to use. The test substances were administered orally one hour before intra-plantar injection of the right hind paw with carrageenan (50 μl of 1% suspension). Hind paw edema, as a measure of inflammation, was recorded 4 hours after carrageenan administration using a plethysmometer with water cell (12 mm diameter).

TABLE V
Group assignment

| | Dose | | | % |
|---|---|---|---|---|
| Description | Per kg | | N | inhibition |
| Control Vehicle | 10 | mL | 5 | 0 |
| (2% Tween 80) | | | | |
| 1 Harpagophytum procumbens:1 Ribes | 125 + | | 5 | 23 |
| nigrum | 125 mg | | | |
| (anti-inflammatory composition 1) | | | | |
| 1 Harpagophytum procumbens:1 Ribes | 83.3 + | | 5 | 18 |
| nigrum:1 Green Shell Mussel | 83.3 + | | | |
| (anti-inflammatory composition 2) | 83.3 mg | | | |
| 1 Harpagophytum procumbens:1 Ribes | 50 + 50 + | | 5 | 27 |
| nigrum:1 Green Shell Mussel:1 Viola | 50 + 50 + | | | |
| tricolour:1 Origanum vulgare | 50 mg | | | |
| (Anti-inflammatory Composition 3) | | | | |
| 1 Harpagophytum procumbens:1 Ribes | 62.5 + | | 5 | 23 |
| nigrum:½ Bromellian:½ Curcumin:1 | 62.5 + | | | |
| Boswellia serrata | 31.25 + | | | |
| (Anti-inflammatory composition 4) | 31.25 + | | | |
| | 62.5 mg | | | |
| Aspirin (positive control) | 150 | mg | 5 | 36 |
| Phenylbutazone (positive control) | 30 | mg | 5 | 23 |
| Celebrex (positive control) | 10 | mg | 5 | 23 |
| Naproxen (positive control) | 30 | mg | 5 | 36 |

As expected, administration of oral compositions of Aspirin at 150 mg/kg, Naproxen at 30 mg/kg, Phenylbutazone at 30 mg/kg, and Celebrex at 10 mg/kg exhibited significant anti-inflammatory activity in the carrageenan-induced paw edema assay in mice. Anti-inflammatory compositions 1, 2, 3 and 4 also exhibit significant anti-inflammatory activity in the model tested. The results clearly demonstrate that compositions comprising both Harpagophytum procumbens, and Ribes nigrum exhibit anti-inflammatory activity. Compositions comprising additional components also exhibit anti-inflammatory activity as shown herein.

### Example 2

### Methods of Treating Adjuvant-Induced Arthritis in Rats

The study was conducted in male Lewis rats obtained from Charles River Japan, Inc. The animals were housed in a controlled temperature (21° C.-24° C.) and humidity (54%-68%) environment with 12 hour light/dark cycles for at least 1 week prior to use. Rats had Laboratory Rodent Diet MF-18 (Oriental Yeast Co., Ltd., Japan) and water (reverse osmosis purified water) ad libitum. The entire study and animal conditions and manipulations were conducted in accordance with the Guide for the Care and Use of Laboratory Animals (National Academy Press, Washington, D.C., 1996).

Eight groups of 5 rats weighing approximately 210±10 g were used. Anti-inflammatory composition 5, Phenylbutazone, Celebrex, Dexamethasone and 2% Tween 80 were administered orally once daily for 5 consecutive days, with the first dose given one hour before Complete Freund's Adjuvant (CFA) challenge. Anti-inflammatory composition 5 was administered at 25 or 60 mg/kg, Phenylbutazone at 35 mg/kg, Celebrex at 10 mg/kg and Dexamethasone at 5 mg/kg. A dosing volume of 10 ml/kg was used. A well-ground suspension of killed Mycobacterium tuberculosis (0.3 mg in 0.1 ml of light mineral oil (CFA) was administered in a single dose into the subplantar region of the right hind paw 1 hour following the first dose of the test and reference compositions.

Right hind paw volume was measured 4 hours after single CFA injection (denoted day 1), and on day 5 (acute phase), while the left hind paw (without CFA) volume was measured on day 14 and 18 (delayed phase). Hind paw volume was measured by plethysmometer and water cell (25 mm diameter).

TABLE VI
Group assignment

| | | | | % inhibition |
|---|---|---|---|---|
| | Dose | | | Day 14-0, |
| Description | Per kg | | N | 18-0, 18-14 |
| Vehicle (2% Tween 80) | 10 | mL | 5 | 0, 0, 0 |
| Anti-inflammatory composition 5 | 25 | mg | 5 | 15, 13, 11 |
| Anti-inflammatory composition 5 | 60 | mg | 5 | 17, 17, 18 |
| Phenylbutazone | 35 | mg | 5 | 18, 18, 18 |
| Celebrex | 10 | mg | 5 | 20, 25, 30 |
| Dexamethasone 21 acetate | 5 | mg | 5 | 66, 61, 56 |

The results provided above suggest that anti-inflammatory composition 5 was capable of reducing inflammation at various concentrations in an adjuvant-induced arthritis assay.

### Example 3

### Mono-Iodoacetate (MIA) Chemically-Induced Osteoarthritis in Rats

A total of seventy (70) male Sprague-Dawley rats plus five (5) spares (Harlan Sprague Dawley, Inc., Indianapolis, Ind., USA) were used. The animals were pathogen free and of approximately 12 weeks of age upon start of experiments. There were 10 animals per group. The animals were individually housed in clear polycarbonate plastic cages but received enrichment in the way of Enrich-o-cobs bedding. The animals were acclimated for a minimum of 7 days prior to the start of the experimental procedures. The temperature was maintained at 18-26° C. (64-79° F.) with a relative humidity of 30-70%. Animals had ad libitum access to certified Pico rodent diet and water (deionized water system).

Animals were allocated to the treatment groups as specified in Table VII based on baseline von Frey results. The mean values of the groups were then reviewed to ensure that the groups were homogeneous.

Animals were first anesthetized with isoflurane and the surgical area was swabbed with chlorohexadine. For the animals in the saline treated group, 50 μl of sterile saline was delivered using a 28-gauge needle inserted through the intra-patellar ligament. Similarly, the animals in the MIA group received 50 μl of a 40 mg/ml solution (i.e. equal to 2 mg/injection) of mono-iodoacetate delivered in a similar fashion. All injections were on the left hind limb.

Body weights were taken one day after arrival, prior to injection of MIA, weekly and then prior to necropsy. Animals were observed daily for signs of ill health and general reaction to experimental procedures and/or treatments. Any significant exceptions to normal healthy appearance and behavior were recorded and detailed in the study records.

Prior to surgery and on days 14, 21, and 28, the animals underwent behavioral testing for tactile allodynia. The behavioral testing occurred within 2.5 hours of dosing.

The animals underwent acclimation to the allodynia procedure on the left foot of the rat. The acclimation to the apparatus occurred 2 to 3 days prior to testing, as this habituated the rats to the testing chamber and allowed the animals to be calm enough to be properly tested.

Prior to surgery, the animals underwent baseline Von Frey testing for mechanical allodynia on their left hindpaw. Any rat with a baseline score below 5 was excluded from the study.

On each test day, rats were placed in the von Frey testing apparatus for a 15-20 min period prior to beginning the testing. Tactile allodynia (i.e. mechanical allodynia) was then determined by applying a series of calibrated nylon filaments through the cage floor against the plantar surface of the left hindpaw. The rats were unrestrained and unhandled during the test. The diameters of the filaments corresponded to a logarithmic scale of force exerted and thus a linear and interval scale of perceived intensity. The withdrawal threshold was determined according to Chaplan's “up-down” method involving the use of successively larger and smaller fibers to determine the 50% withdrawal threshold. Briefly, when the rat lifts its paw in response to the pressure, the filament size was recorded and a weaker filament was used next. Conversely, in the absence of a response, a stronger stimulus was used. A sequence of such responses was thereby generated and the 50% response threshold was calculated using a response variable spreadsheet. Significant differences in tactile allodynia were based on the comparison of group mean values.

The animals were dosed as described in Table VII. The dosing occurred within one hour of formulation, at a volume of 5 mL/kg.

The animals receiving test compositions were dosed daily by oral gavage beginning one day post-MIA or saline injection. On days of behavioral testing, the behavioral testing occurred 2 hours±30 minutes after dosing.

The day after the last behavioral test, the animals were humanely euthanized by CO₂ asphyxiation.

TABLE VII
Treatment Groups

| | Test Article/ | Route/ | Dose | |
|---|---|---|---|---|
| Description | Physical Description | Frequency | mg/kg | n |
| IA Vehicle | Vehicle | PO-QD/ | 0 | 10 |
| (no MIA) | 2% Tween 80 | d1-28 | | |
| 2 mg | Vehicle | PO-QD/ | 0 | 10 |
| MIA IA | 2% Tween 80 | d1-28 | | |
| 2 mg | Glucosamine-Chondroitine | PO-QD/ | 45 | 10 |
| MIA IA | Cream tablet | d1-d28 | | |
| 2 mg | Anti-inflammatory | PO-QD/ | 150 | 10 |
| MIA IA | composition 5 | d1-d28 | | |

MIA—Mono-iodoacetate
IA—Intra-articular injection

Anti-inflammatory composition 5 and glucosamine-chondroitine were administrated beginning on study day 1 to assess if these product mixtures could reduce the progression or severity of the osteoarthritis. By day 28, administration of the glucosamine-chondroitine mixture to rats had not caused a reduction in the severity of pain (i.e., prevention of articular damage). However, anti-inflammatory composition 5 caused a reduction in the severity of pain and therefore appears to have reduced the degree of articular damage. A comparison of the means was also performed for this data using the t-test (alpha=0.05). There was no statistical difference between MIA-vehicle control group and a composition comprising glucosamine-chondroitine (p=0.54) suggesting that the administration of glucosamine-chondroitine did not reduce the severity of pain or the degree of articular damage. However, a comparison of the means between results obtained with anti-inflammatory composition 5 and a composition comprising glucosamine-chondroitine did reveal that these two groups were statistically different (p=0.04). Similarly, a comparison of the means between results obtained with mono-iodoacetate alone versus monoiodoacetate in combination with anti-inflammatory composition 5 indicated a statistical difference (p value of 0.06) thereby indicating that anti-inflammatory composition 5 reduced inflammation, the severity of pain and/or the degree of articular damage.

### Example 4

### Effect of Anti-Inflammatory Composition #7 in Treating a Patient Exhibiting Lumbar Pain and Chronic Lumbar Degenerative Bone Disease (Osteoarthrosis)

Capsules comprising anti-inflammatory composition #7 were prepared (see Table IV. The capsules also comprise as non-medicinal ingredients vanillin, microcrystalline cellulose, silicon dioxide, maltodextrin, glyceryl bihenate, hydrogenated cotton seed oil, and magnesium stearate.

A 52 year old female patient with no relevant medical history except for a history of chronic lumbar pain consulted her physician with incapacitating acute lumbar pain that confined her to bed for several days. An X-ray of the spinal cord resulted in a diagnosis of degenerative lumbar bone disease (i.e., decrease in the intervertebral space). No other diagnostic procedures were performed.

The patient was treated with non steroidal anti-inflammatory drugs (NSAIDs) for pain and underwent physiotherapy for two years.

Subsequently, the patient suffered intermittent relapses of pain several times a month, although the pain was of lower severity than previously experienced. To relieve the pain, the patient took Ibuprofen and on occasion had to rest in bed.

Physical activities as well as the patient's work as an esthetician (a profession that does not always favor an ergonomic position that is desirable for the lower back) are factors that contributed to relapses of acute pain and required analgesic therapy.

The patient began taking 2 capsules of anti-inflammatory composition #7 (1000 mg) in the morning and evening when the pain was present and in order to prevent the occurrence of acute pain prior to performing physical activities such as gardening or sports. The patient claims that the pain is substantially reduced by taking the composition. Further, the composition appears to be capable of preventing relapse of acute pain. No other adverse events were reported by the patient and the patient does not require any concomitant analgesic therapy.

### Example 5

### Effect of Anti-Inflammatory Composition #7 in Treating a Patient Exhibiting Gonalgia Associated with a Traumatic Fissure of the Medial Patella Cartilage and of the Meniscus

A 53 year old athletic patient with no medical history other than multiple fractures (right hand, ribs, nose) subsequent to sport injuries and a car accident was tested for the effect of anti-inflammatory composition #7. This patient actively practiced tennis and practices equestrian jumping at a frequency of 5 to 6 one hour sessions per week.

Approximately 1 year ago, the above patient experienced acute right gonalgia after equestrian riding. He stopped equestrian jumping for a period of time, but continued to experience intense pain in his right knee after exertion. He underwent several sessions of physiotherapy and subsequently underwent an MRI of the right knee. The MRI scan revealed a small fissure of the medial facet cartilage involving the subchondral bone, but without any change to the bone. He also had a horizontal inferior tear of 1.8 cm that involved the cornus and the meniscus, in addition, to a radial tear of 5 mm. This is associated with edema of the bone marrow of the tibial plateau.

Based on results obtained from the MRI scan, the patient's orthopedist recommended a surgical approach to manage his condition. However, the patient did not want to stop equestrian jumping and instead chose a therapeutic approach. The patient tried several NSAIDs but each of these was associated with gastrointestinal intolerance including abdominal pain and constipation, which lead to the patient to stop taking the medication. Sessions of physiotherapy were maintained and after several months of rehabilitation he returned to equestrian jumping. However, this physical activity would, on occasion, cause intolerable pain to the patient's right knee and this would force the patient to stop the activity.

As a result of the patient's intolerance to NSAIDs, the patient elected to take anti-inflammatory composition #7 and Artriphen, a non-plant based composition comprising glucosamine. The patient consumed 2 capsules of anti-inflammatory composition #7 and 2 capsules of Artriphen daily and experienced a significant improvement of his symptoms and was able to return to equestrian jumping at a more active and intense frequency.

### Example 6

### Effect of Anti-Inflammatory Composition #7 in Treating a Patient Exhibiting a L4-L5 Disc Hernia and a Congenitally Small Lumbar Channel

A 45 year old male patient complained about lower back pain along with pain in the hips, lower legs and ankles radiating down to the toes, with numbness on the skin surface of the right thigh. The patient could not bend forward or lift the left or right leg without experiencing significant pain in these extremities and the lower back. The patient could only walk short distances without having to sit down due to pain in the extremities and lower back, and could not lift objects of more than 5 pounds without experiencing substantial increases in pain of the lower back. At night, the pain prevented the patient from sleeping.

CAT and MRI scans revealed that the patient had a congenital small lumbar channel due to small pedicles. At L4-L5, a central disc hernia causing spinal stenosis, a small to moderate reduction in the size of the spinal channel was observed.

The patient's pain was not relieved when treated with 400 mg ibuprofen (every 4-6 hours), 4000 mg Tylenol daily, or 100 mg or 200 mg Celebrex twice a day. The pain is only partially relieved by administering 500 mg Naprosyn twice a day. Relief during severe episodes of pain was achieved with 4 mg hydromorphone twice a day with or without 10 mg cyclobenzaprine (muscle relaxant). The hydromorphone and cyclobenzaprine were taken mainly at night for sleep and almost every 3 days to help reduce the intolerable pain during daily activities.

In an attempt to lessen the patient's pain, the patient consumed anti-inflammatory composition #7 twice daily for 2 months. During this period, the patient significantly reduced his intake of hydromorphone and muscle relaxant. Anti-inflammatory composition #7 when taken twice daily, appeared to provide adequate relief of the pain. During this time, the patient's pain in the lower extremities (including ankle) and lower back were significantly reduced allowing the patient to function relatively normally but not perform excessive physical activities. For pain flare ups during the day, the patient took an additional dose of anti-inflammatory composition #7 to reduce the pain. The patient did not experience any gastrointestinal disturbances during this period.

### Example 7

### Veterinary Applications of the Anti-Inflammatory Compositions as Described Herein

Case Number 1:

A 10 year old Husky with a history of bilateral rupture of the cruciate ligament had bilateral knee surgery to correct this problem. The animal was also known to have zinc deficiency.

The dog was prescribed metacam for knee pain associated with arthrosis. The dog exhibited symptoms of stiffness and limping. The pain was only partially controlled with metacam and the dog exhibited signs of intolerance to the medication.

The dog was administered anti-inflammatory composition #8 comprising 60 mg Harpagophytum procumbens (Devil's claw) root extract standardized to about 10% harpagosides, 60 mg Ribes nigrum (Black Currant) leaf or seed extract standardized to about 1% rutines, 180 mg Boswellia seratta (Boswellia) extract standardized to a minimum of 69% boswellic acids, 40 mg DHA-EPA, 35 mg Cucurma longa (Tumeric) extract standardized to a minimum of 95% curcumin, 40 mg Bromelain (Ananas comosus) extract standardized to a minimum 2500 GDU (Gelatin Dissolving Units), 15 mg hyaluronic acid, L-glutamine (30 mg), chondroitin (60 mg), methylsulfonylmethane (90 mg) and glucosamine (300 mg).

The animal was administered anti-inflammatory composition #8 in a dosage of 1 gram per 10 kg body weight daily and after a few weeks the owner saw improvement in the mobility of the dog. The owner also indicated that the dog was appeared healthier and happier. Clinical examination revealed significant improvement in pain relief with no evidence of intolerance to the plant extract anti-inflammatory composition.

Case Number 2:

A 12 year old Airedale known with symptoms of arthrosis in the back was started on anti-inflammatory composition #8. The animal was administered a dosage of 1 gram per 10 kg body weight.

The owner had noted that before taking the plant extract anti-inflammatory composition, the dog showed signs of stiffness and pain in the back, specifically, the dog was reluctant to be touched on the back.

After taking the plant extract anti-inflammatory composition #8 daily for 10 to 14 days, the severity of symptoms described above improved and the owner was satisfied.

The results described in the examples suggest that the compositions comprising plant extracts as provided herein can be used to prevent and/or treat inflammation or conditions such as pain that result from inflammation.

All citations are hereby incorporated by reference.

The present invention has been described with regard to one or more embodiments. However, it will be apparent to persons skilled in the art that a number of variations and modifications can be made without departing from the scope of the invention as defined in the claims.

### CITATIONS

1. Nathan, C. 2002. Points of control in inflammation. Nature 420:846-852.
2. Abbas, A., and A. Lictman. 2003. In Cellular and Molecular Immunology. L. Goldman, and D. Ausiello, eds. Saunders, Philadelphia. 16-39.
3. Collins, T. 1999. In Robbins Pathologic Basis of Disease. R. Cotran, V. Kumar, and T. Collins, eds. Saunders, Philadelphia. 50-88.
5. Tak, P. P., and B. Bresnihan. 2000. The pathogenesis and prevention of joint damage in rheumatoid arthritis: advances from synovial biopsy and tissue analysis. Arthritis and rheumatism 43:2619-2633.
6. Firestein, G. S. 2003. Evolving concepts of rheumatoid arthritis. Nature 423:356-361.
7. Beale, B. S. 2004. Use of nutraceuticals and chondroprotectants in osteoarthritic dogs and cats. Vet Clin North Am Small Anim Pract 34:271-289, viii.
8. Aragon, C. L., E. H. Hofmeister, and S. C. Budsberg. 2007. Systematic review of clinical trials of treatments for osteoarthritis in dogs. J Am Vet Med Assoc 230:514-521.
9. Henrotin, Y., C. Sanchez, and M. Balligand. 2005. Pharmaceutical and nutraceutical management of canine osteoarthritis: present and future perspectives. Vet J 170:113-123.

What is claimed is:

1. A composition comprising Harpagophytum procumens (Devil's claw) extract and Ribes nigrum (Black Currant) extract.

2. The composition of claim 1, wherein the Harpagophytum procumens (Devil's claw) extract comprises about 5% to 20% harpagosides, and the Ribes nigrum extract comprises between about 0.1% to 2% rutines.

3. The composition of claim 1, wherein the composition further comprises one or more components or extracts of Perna canaliculus (Green Shell Mussel), Salix Alba (White Willow), Tanacetum Parthenium (Feverfew), Equisetum arvense (Horsetail), Spireae ulmaria (propwort), Betula alba (Birch), Urtica dioica (Stinging Nettle), Solidago virgaurea (Goldenrod), Bosellia seratta (Boswellia), Curcumin longa (Tumeric), Bromelain (Ananas comosus), Griffonia simplicifolia, Petasites hybridus (Butterbur), marine algae, or a combination thereof.

4. The composition of claim 1, wherein the composition further comprises fish meal, type II collagen, glucosamine, DHA, EPA, hyaluronic acid, L-glutamine, chondroitin, methylsulfonylmethane, dextrose, whey protein, minerals including, without limitation calcium, phosphate, manganese, magnesium, zinc, copper (free, chelated or both), vitamin B12, vitamin E, Vitamin C, beta carotene, microcrystalline cellulose, polyethylene glycol, starch, stearin, hydrogenated oils, talc, stearate or a combination thereof.

5. The composition of claim 1 comprising Harpagophytum procumbens (Devil's claw) extract, Ribes nigrum (Black currant) extract, Perna canaliculus (Green Shell Mussel), Salix Alba (White Willow) extract, and Tanacetum Parthenium (Feverfew) extract.

6. The composition of claim 5, comprising 250 mg Harpagophytum procumens (Devil's claw) extract standardized to about 10% harpagosides, 100 mg Ribes nigrum (Black currant) extract standardized to about 1% rutines, 250 mg Perna canaliculus (Green-Shell mussel) standardized to about 20% omega-3 polyunsaturated fatty acids, 100 mg Salix alba standardized to about 10% salicin and Tanacetum parthenium (Feverfew) extract standardized to 0.2% parthenolide.

7. The composition of claim 1 further comprising Salix alba (White Willow) extract, Tanacetum Parthenium (Feverfew) extract, Boswellia seratta (Boswellia) and optionally DHA and/or EPA.

8. The composition of claim 1 further comprising Boswellia seratta (Boswellia), DHA and/or EPA, Cucurma longa (Tumeric), Bromelain (Ananas comosus) and optionally one or more of hyaluronic acid, L-glutamine, chondroitin, methylsulfonylmethane and glucosamine.

9. The composition of claim 1, further comprising fish meal/collagen type II, Glucosamine sulphate, marine algae, methylsulfonylmethane, chondroitine sulphate, Bromelia (pineapple extract), curcumin, L-glutamine, hyaluronic acid, dextrose, DHA complex, whey protein, manganese chelate, manganese sulphate, zinc chelate, Zinc oxide, Vitamin B12, Vitamin B6, Vitamin B9, Copper chelate, Cupric sulphate, Vitamin E, Vitamin C, Beta carotene, Dextrose, Equisetum arvensis, Boswellia serata, Harpagophytum procumbens, and Ribes nigrum.

10. The composition of claim 1 comprising Devil's claw, Green shell mussel powder, Ribes nigrum, Viola tricolour, Zea maize, Origanum vulgare, Dicalcium phosphate, Microcrystalline cellulose E460, Polyethylene glycol, Starch, Stearin, Glyceryl dibehenate, Hydrogenated cottonseed oil, Talc, and Magnesium stearate.

11. A method of preventing, treating or both preventing or treating inflammation or a condition that results from inflammation in a subject comprising the step of administering the composition of claim 1 to the subject in need thereof.

12. The method of claim 11, wherein the inflammation or condition that results from inflammation is arthritis.

13. The method of claim 12, wherein the arthritis is rheumatoid arthritis or osteoarthritis.

14. The method of claim 11, wherein the subject is concurrently treated with a steroid, non steroidal anti-inflammatory drug, narcotic, muscle relaxant or any combination thereof.

15. The method of claim 11, wherein the condition that results from inflammation is pain.

16. The method of claim 11, wherein the subject is a mammalian subject.

17. The method of claim 16, wherein the subject is a human subject.

18. The composition of claim 1, further comprising at least one other therapeutic compound or composition for treatment of inflammation, pain, or a combination thereof.
